(19) United States
(12) Patent Application Publication
(10) Pub. No.: US 2007/0190148 A1
(43) Pub. Date: Aug. 16, 2007

(54) Gel compositions, apparatuses and fluid separation methods

(76) Inventors: Peter Cronin, Newark, DE (US); Daniel J. Halbert, Hockessin, DE (US); Richard J. Spontak, Raleigh, NC (US); Bin Wei, Somerville, NJ (US)

Correspondence Address: DADE BEHRING INC.;LEGAL DEPARTMENT, 1717 DEERFIELD ROAD, DEERFIELD, IL, 60015, US

(21) Appl. No.: 11/353,901

(22) Filed: Feb. 14, 2006

(51) Int. Cl. A61K 9/14

(52) U.S. Cl. 424/484; 977/906

(57) ABSTRACT

Compositions including a liquid or liquid-like carrier and first and second particles dispersed therein. The first and second particles differ from each other in at least one surface chemistry property, such as hydrophobicity. The gel compositions can be used in a variety of applications, including the separation of blood samples into component fractions. Processes for producing gel compositions having desirable properties are also provided.

### FIELD OF THE INVENTION

The invention relates generally to gel compositions, processes for making gel compositions, apparatuses including gel compositions, and methods for separating fluid samples into component fractions. Gel compositions and other aspects of the invention are useful in a variety of applications, including separation of blood samples in the clinical laboratory.

### BACKGROUND OF THE INVENTION

Physical gels include solid particles suspended in a liquid or liquid-like carrier. Mechanical and chemical interactions between the components give structure to the gel and typically provide a composition that is more viscous than the carrier alone.

Physical gels are used in a variety of applications. For example, it is now widely known and accepted to use a gel composition to achieve separation of blood samples into serum and clot fractions. By using a gel having a specific gravity between that of the serum and clot fractions of the blood, a whole blood sample can be separated into its component fractions using standard centrifugation techniques. During centrifugation, a properly selected gel composition migrates to a position between the serum and clot fractions due to its intermediate specific gravity. This migration produces a physical barrier between the components, which greatly facilitates the use of one or both components in subsequent analyses. Examples of known gel compositions for use in this manner are described in U.S. Pat. No. 3,852,194 to Zine for an APPARATUS AND METHOD FOR FLUID COLLECTION AND PARTITIONING and U.S. Pat. No. 4,049,692 to Zine for a STABILIZED BLOOD SEPARATING COMPOSITION, each of which is hereby incorporated into this disclosure in its entirety.

Despite the advantages offered by the use of gels in a variety of applications, the need for improvement still exists. For example, gels are often susceptible to becoming increasingly liquid-like over time. As a gel begins to flow, that is, as it begins to demonstrate more liquid-like characteristics, its effectiveness as a barrier diminishes. This loss of viscosity is particularly problematic for gels used in the separation of blood samples. Described as “wet out,” the transition toward a flowable liquid eventually leads to a breakdown of the barrier between the serum and clot fractions in a blood sample. Wet-out can be particularly problematic when the container holding the separated sample is moved, intentionally or accidentally. Physical movement of the container can exacerbate the wet-out problem to a point at which the fractions and/or components of the fractions re-mix, which may affect subsequent analyses conducted on the sample or prevent the performance of such analyses altogether.

Accordingly, a need exists for improved gel compositions, processes for making gel compositions, apparatuses that include gel compositions, and methods for separating fluid samples into component fractions.

### SUMMARY OF EXEMPLARY EMBODIMENTS OF THE INVENTION

The invention provides gel compositions useful in a variety of applications. In one exemplary application, gel compositions according to the invention are used in the separation of blood samples into serum and clot fractions.

Gel compositions according to the invention include a liquid or liquid-like carrier with at least two kinds of particles dispersed therein. The first and second particles of these at least two kinds of particles differ from one another in at least one surface property, such as hydrophobicity.

In gel compositions according to exemplary embodiments of the invention, a silicone fluid is used as the carrier.

In gel compositions according to exemplary embodiments of the invention, at least one of the first and second particles comprises a nanoparticle. In gel compositions according to some exemplary embodiments, both the first and second particles comprise nanoparticles. In some exemplary embodiments of the invention, the first and second nanoparticles comprise fumed silica compositions. In gel compositions according to some exemplary embodiments of the invention, the second nanoparticle comprises a fumed silica composition that has been treated to increase hydrophobicity. In one particular embodiment, the second nanoparticle comprises a relatively hydrophobic derivative of the first nanoparticle.

The at least two kinds of particles can be present in the gel composition at a variety of total particle concentrations. In exemplary embodiments, the first and second particles are present at a total particle concentration of less than about 10 weight percent of the gel composition. The second particle can comprise any suitable fraction of the total particle concentration. In exemplary embodiments, the second particle comprises greater than about 50 weight percent of the total particle concentration. In other exemplary embodiments, the second particle comprises greater than about 60 weight percent of the total particle concentration, greater than about 70 weight percent of the total particle concentration, greater than about 85 weight percent of the total particle concentration, and greater than about 95 weight percent of the total particle concentration.

A gel composition according to one exemplary embodiment of the invention comprises first and second nanoparticles dispersed in a silicone fluid. In this embodiment, the second nanoparticle is more hydrophobic than the first nanoparticle.

The invention also provides apparatuses useful in the separation of a fluid sample into component fractions having different densities. One apparatus according to an exemplary embodiment of the invention comprises a vessel defining an interior space and a gel composition according to the invention disposed in the interior space. An apparatus according to another exemplary embodiment of the invention comprises a vessel defining first and second interior spaces separated by a gel composition according to the invention that comprises an annular body that separates the interior space into first and second chambers.

The invention also provides methods of separating a fluid into component fractions having different densities. An exemplary method according to the invention comprises providing a vessel that defines an interior space and has a gel composition disposed in the interior space; disposing the fluid to be separated in the interior space adjacent the gel composition; and the vessel at a velocity sufficient to cause moving at least one component fraction of the fluid sample to move through the gel composition to effect separation from at least one other component fraction of the fluid sample. Methods according to the invention are particularly well-suited for use in the separation of clinical samples, such as blood samples.

Additional understanding of the invention can be gained with review of the following detailed description of exemplary embodiments of the invention.

### BRIEF DESCRIPTION OF THE DRAWINGS

FIG. 1 is a sectional view of an apparatus according to an exemplary embodiment of the invention.

FIG. 2 is a sectional view of the apparatus illustrated in FIG. 1 following a fluid separation method.

FIG. 3 is a perspective view of an apparatus according to a second exemplary embodiment of the invention.

FIG. 4 is a top view of the apparatus illustrated in FIG. 3.

FIG. 5 is a sectional view, taken along line 5-5, of the apparatus illustrated in FIG. 3.

### DETAILED DESCRIPTION OF EXEMPLARY EMBODIMENTS OF THE INVENTION

The following detailed description describes exemplary embodiments of the invention for the purpose of enabling one of ordinary skill in the relevant art to make and use the invention. The detailed description is not intended to limit the invention, or its protection, in any manner.

As used herein, the term “gel composition” refers to a composition according to the invention. The descriptor “gel” refers to the physical properties of the compositions according to the invention, which are generally semi-solid systems that include a liquid or liquid-like component and solid particles dispersed therein.

The invention provides gel compositions that are useful in a variety of applications. In one exemplary utility, gel compositions according to the invention can be used in the separation of fluids into component fractions, such as the separation of blood samples into serum and clot fractions. Compositions according to the invention are particularly well-suited for use in the evaluation of blood samples in a clinical laboratory setting, such as in the diagnosis of disease based on analyses conducted on a blood sample fraction. The compositions can readily be used with analysis equipment typically employed in the clinical laboratory, such as the Stratus® CS Analyzer sold by Dade Behring (Deerfield, Ill.).

Gel compositions according to the invention comprise at least two kinds of particles dispersed in a liquid or liquid-like carrier. The first and second particles of these particles comprise different chemical entities that differ in at least one surface chemistry property. The particles can differ on any suitable surface chemistry property, and on any number of surface chemistry properties. Examples of suitable surface chemistry properties for which a difference between particles is advantageous include, but are not limited to, hydrophobicity, solubility, electrostatic properties, and ionic properties.

One of the first and second particles can be a derivative of the other. That is, one of the particles can be a product of a chemical treatment or other process conducted on the other particle to produce a different chemical entity having a desired surface chemistry property that is different than that property exhibited by the other particle. For example, the second particle can comprise a derivative of the first particle that is the product of a treatment for increasing hydrophobicity, such as treatment with a dimethyl silicone fluid.

The first and second particles can comprise any suitable particles and need only be capable of being dispersed in the carrier. The particles chosen for any particular gel composition according to the invention will depend on several factors, including the desired viscosity of the gel composition and the application for which the gel composition is intended.

One or more of the particles can comprise a nanoparticle. As used herein, the term “nanoparticle” refers to a solid particle that has a diameter of less than 100 nm. In some embodiments the first and/or second nanoparticle will have a diameter of less than 50 nm. In other embodiments, the first and/or second nanoparticle will have a diameter of less than 25 nm, 10 nm, 5 nm, 2 nm, or 1 nm. The use of nanoparticles in gel compositions according to the invention is considered advantageous at least because of the ability of these particles to be dispersed throughout a carrier. With larger particles, it can be difficult to achieve the loads necessary to obtain desired characteristics of the resulting gel composition.

Any suitable nanoparticle can be used in the gel compositions according to the invention. If nanoparticles are used, the only limitation on the selection of nanoparticles is that the first and second particles must comprise different chemical entities that differ in at least one surface chemistry property. The specific nanoparticle(s) chosen for any particular composition will depend on several factors, including the desired stiffness and overall mechanical strength of the gel composition. The inventors have determined that fumed silica nanoparticles are advantageously used in the gel compositions according to the invention. Fumed silicas are considered advantageous at least because of their extremely small particle size, relatively large surface area and ready availability. These advantages allow these nanoparticles to be readily incorporated into carriers, such as silicone fluids, and to influence the overall mechanical strength and rheological properties of the gel compositions according to the invention.

Examples of suitable nanoparticles for use in the gel compositions according to the invention include the CAB-O-SIL® fumed silica products available from Cabot Corporation (Boston, Mass.). One particularly advantageous pair of nanoparticles comprises CAB-O-SIL M-5 untreated fumed silica and CAB-O-SIL TS-720 treated fumed silica. TS-720 is a derivative of M-5 in that it has undergone treatment with dimethyl silicone fluid to increase its hydrophobicity over that of the M-5 nanoparticle. The treatment replaces many of the surface hydroxyl groups on the M-5 fumed silica with a polydimethyl siloxane polymer, making the TS-720 extremely hydrophobic.

Each of the first and second particles is dispersed in the carrier at a particular weight percent (weight of particle/weight of carrier). Together, the first and second particles are present in the composition at a total particle concentration ((weight of first particle+weight of second particle)/weight of entire composition). Any suitable total particle concentration can be used, and the specific total particle concentration chosen in a particular gel composition according to the invention will depend on several factors, such as the desired viscosity of the gel composition. Furthermore, each particle can comprise any suitable portion of the total particle concentration. Indeed, the fraction of the total particle concentration for a given particle or particles can be manipulated to optimize characteristics of a gel composition according to the invention. For example, as explained more fully below, the inventors have determined that gel compositions having desirable characteristics for use in blood separation procedures are produced when a relatively hydrophobic nanoparticle comprises a particular fraction of the total particle concentration.

Any suitable liquid or liquid like material can be used as the carrier in a gel composition according to the invention. The selection of an appropriate carrier can be based on a variety of factors, including the viscosity of the carrier alone and the ability of the selected particles to be dispersed within the carrier. Examples of suitable carriers include, but are not limited to, silicone fluids and oligoethers, such as polyethylene glycol (PEG) and polypropylene glycol (PPG). Silicone fluids are considered particularly advantageous for use in gel compositions according to the invention due at least in part to their ready availability from commercial sources.

For gel compositions in which a silicone fluid is used as the carrier, any suitable silicone fluid can be used. The specific silicone fluid chosen for any particular composition will depend on several factors, including the desired final specific gravity of the composition and other considerations. The inventors have determined that polydimethylsiloxane fluids are advantageously used in the gel compositions according to the invention. The various polydimethylsiloxane fluids available from Dow Corning (Midland, Mich.; offered as the Dow Corning® 200 Fluids) are suitable for use in the gel compositions according to the invention and are considered advantageous at least due to their ready availability and wide range of available viscosities. These fluids are considered particularly advantageous for use in gel compositions for the separation of blood samples due at least in part to their accepted biocompatibility.

The invention also provides processes for making gel compositions having desirable properties. An initial step of a process according to the invention comprises selecting a liquid or liquid-like carrier. Another step of the process comprises selecting a first particle. Another step of the process comprises selecting a second particle that differs from the first particle in at least one surface chemistry property. Another step of the process comprises determining an effective amount of the first particle to achieve a desired property for a gel composition. Another step of the process comprises determining an effective amount of the second particle to achieve a desired property for a gel composition. Another step of the process comprises dispersing the effective amount of the first particle in the carrier. Another step of the process comprises dispersing the effective amount of the second particle in the carrier.

In preparing gel compositions according to the invention, using the process above or otherwise, the first and second particles are advantageously mixed into the carrier until each of the particles is substantially evenly distributed throughout the carrier. Any suitable mixing technique can be employed to prepare the gel compositions in this manner. The inventors have determined that, in preparing gel compositions according to exemplary embodiments of the invention, the use of a high-shear mixer, such as the SL2T Laboratory Mixer available from Silverson (United Kingdom), facilitates dispersion of nanoparticles in a silicone fluid.

The invention also provides apparatuses useful in the separation of a fluid or fluids into component fractions that have different densities. Apparatuses according to the invention comprise a vessel defining an interior space and a gel composition according to the invention disposed in the interior space. Any gel composition according to the invention can be used and the specific gel used in any particular apparatus according to the invention will depend on several factors, including the relative densities of the component fractions of the fluid for which the apparatus is intended to separate, as described above.

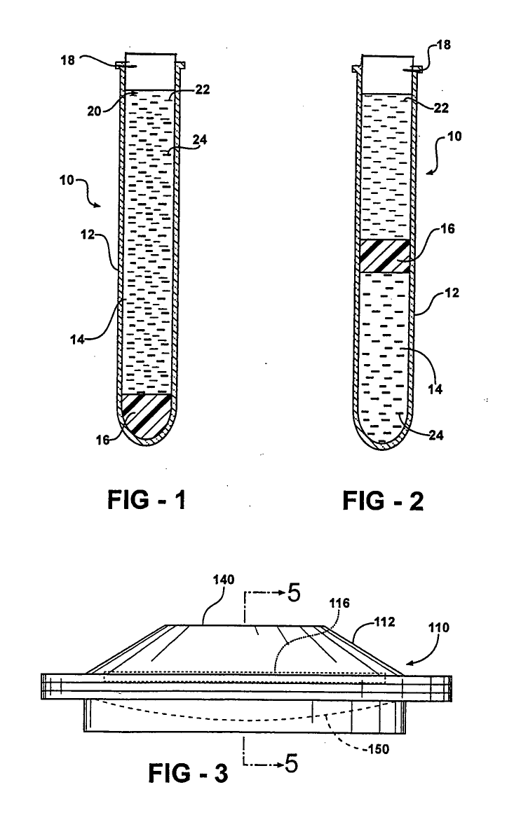

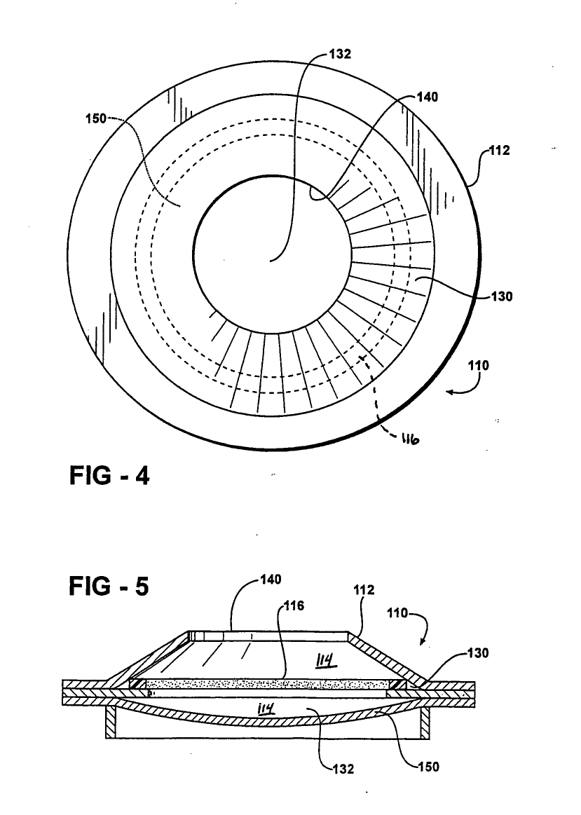

FIGS. 1 and 2 illustrate an apparatus 10 according to a first exemplary embodiment of the invention. In this embodiment, the vessel 12 comprises a cuvette, such as a laboratory test tube. The vessel 12 defines an interior space 14 within which gel composition 16 according to the invention is disposed. The vessel 12 can include a closure 18 or other suitable means for separating the interior space 14 from the external environment.

In use, a fluid 20 is initially disposed in the interior space 14 adjacent the gel composition 16. The fluid 20 may have component fractions 22, 24 that have different relative densities. For example, if a blood sample is being used, the fluid 20 comprises a cellular fraction 24 having relatively high density and a plasma fraction 22 with a relatively low density. FIG. 2 illustrates the vessel 10 following its use in a fluid separation method. The gel composition 16, due to its chosen intermediate density, has migrated to a position in the vessel 10 between the first 22 and second component fractions 24. Component fraction 24, which has a greater density, is disposed at the bottom of the vessel 10 and component fraction 22, which has a lower density, is disposed at the top of the vessel 10. The gel composition 16 is disposed between the fractions and acts as a barrier to substantially prevent subsequent mixing of the separated fractions 22, 24.

FIGS. 3 though 5 illustrate an apparatus 110 according to another exemplary embodiment of the invention. In this embodiment, the vessel 112 comprises a rotor that defines an interior space 114 (FIG. 5) within which a gel composition 116 according to the invention is disposed. The gel composition 116 comprises an annular body that separates the interior space 114 into first 130 and second 132 chambers. The first chamber 130 is disposed radially outward of the gel composition 116 while the second chamber 132 is disposed radially inward of the gel composition 116. An upper opening 140 provides access to the interior space 114. Also, as best illustrated in FIGS. 3 and 5, the second chamber 132 can define a sloped surface 150 that facilitates collection of a fluid fraction from the second chamber 132 following a separation method.

In use, a fluid to be separated can be passed through the opening 140 and disposed in the second chamber 132 of the interior space 114. The vessel 110 is then caused to move, such as by rotation, at a velocity sufficient to force a fraction of the fluid having a relatively high density to move through the gel composition 116 and into the first chamber 130. At this point, the gel composition 116 is disposed between component fractions of the fluid and acts as a barrier to substantially prevent subsequent mixing of the separated fractions.

The invention also provides methods of separating a fluid into component fractions having different densities. An exemplary method according to the invention comprises the initial step of providing a vessel that defines an interior space and includes a gel composition according to the invention disposed in the interior space. Any suitable gel composition according to the invention can be used, and the specific gel composition chosen for the separation of a particular fluid will depend on several factors, including relative densities of the component fractions of the fluid as described above. An additional step comprises disposing the fluid to be separated in the interior space adjacent the gel composition. An additional step comprises moving the vessel at a velocity sufficient to cause at least one component fraction of the fluid sample to move through the gel composition within the vessel to a new position based on a relative density. While any suitable movement can be used in the moving step, rotational movement, such as that achieved by centrifugation, is particularly well-suited for this part of the method. The velocity and duration of the movement in the moving step will depend on several considerations, including the nature of the fluid being separated, the relative densities of the component fractions being separated, and the nature of the gel composition being used. For the separation of blood samples into cellular and plasma fractions, the inventors have determined that rotational movement at about 22,000 revolutions per minute for less than about 5 minutes is sufficient to effect separation of a blood sample into cellular and plasma fractions using gel compositions according to the invention. This can readily be achieved with analysis equipment typically available in the clinical laboratory, such as the Stratus® CS Analyzer sold by Dade Behring (Deerfield, Ill.).

The particles can be incorporated into the silicone fluid at any suitable total nanoparticle concentration and the specific total nanoparticle concentration selected for a particular gel composition will depend on several factors, including the chemical and physical properties of the nanoparticles. In one embodiment of the invention, a total nanoparticle concentration of less than about 10 weight percent of the gel composition provides desirable characteristics. A total nanoparticle concentration within this range provides the desired properties of the gel composition and is readily prepared using standard mixing techniques and equipment. It is noted that each of the nanoparticles can be present at any suitable weight percent within the parameters of the total nanoparticle composition. In other embodiments of the invention, the total nanoparticle concentration is between about 3 and about 7 weight percent of the composition possess desirable properties.

### EXAMPLE

### Preparation of gel compositions

Gel compositions are prepared by mixing M-5 and TS-720 nanoparticles into Dow Corning 200 silicone fluid in a Silverson SL2T high-shear mixer operated at 1500 rpm for approximately 5 minutes or until the nanoparticles are substantially evenly distributed throughout the silicone fluid.

The foregoing disclosure includes the best mode of the inventors for practicing the invention. It is not a complete disclosure of all embodiments of the invention, but rather a detailed description of exemplary embodiments. It is apparent that those skilled in the relevant art will recognize variations of the invention that are not described herein. While the invention is defined by the appended claims, it is not limited to the literal meaning of the claims but also includes these variations.

What is claimed is:

1. A gel composition compatible with blood, said gel composition comprising:
a silicone fluid;
at least two kinds of particles dispersed in the silicone fluid, at least of one said at least two kinds of particles being a first nanoparticle, one of said at least two kinds of particles being more hydrophobic than another of said at least two kinds of particles.

2. A gel composition according to claim 1, wherein said at least two kinds of particles each comprise fumed silica compositions.

3. The gel composition of claim 2, wherein said at least two kinds of particles includes a second nanoparticle.

4. A gel composition according to claim 3, wherein the second nanoparticle comprises a fumed silica composition that has been treated with a dimethyl silicone fluid to increase hydrophobicity.

5. A gel composition according to claim 3, wherein the first and second nanoparticles are present at a total nanoparticle concentration of less than about 10 weight percent of said gel composition.

6. A gel composition according to claim 3, wherein the second nanoparticle comprises greater than about 50 weight percent of the total nanoparticle concentration.

7. A gel composition according to claim 3, wherein the second nanoparticle comprises greater than about 95 weight percent of the total nanoparticle concentration.

8. A gel composition according to claim 3, wherein the first and second nanoparticles are present at a total nanoparticle concentration of between about 3 and about 7 weight percent of said gel composition.

9. A gel composition compatible with blood, said gel composition comprising:
a silicone fluid;
a hydrophilic fumed silica nanoparticle dispersed in the silicone fluid; and
a hydrophobic fumed silica nanoparticle dispersed in the silicone fluid.

10. The gel composition of claim 9, wherein the hydrophilic and hydrophobic fumed silica nanoparticles are present at a total nanoparticle concentration of less than about 10 weight percent of said gel composition.

11. A gel composition according to claim 9, wherein the hydrophobic fumed silica nanoparticle comprises a derivative of the hydrophilic fumed silica nanoparticle.

12. A gel composition according to claim 9, wherein the hydrophobic fumed silia nanoparticle comprises greater than about 50 weight percent of the total nanoparticle concentration.

13. A gel composition according to claim 9, wherein the hydrophobic fumed silica nanoparticle comprises greater than about 95 weight percent of the total nanoparticle concentration.

14. An apparatus for separating a fluid sample into component fractions having different densities, said apparatus comprising:
a vessel defining an interior space; and
a gel composition disposed in the interior space, the gel composition comprising a silicone fluid;
at least two kinds of particles dispersed in the silicone fluid, at least of one said at least two kinds of particles being a nanoparticle, said at least two kinds of particles differing from one another in their surface chemistry properties.

15. The apparatus of claim 14, wherein one of said at least two kinds of particles is more hydrophobic than another of said at least two kinds of particle.

16. The apparatus for of claim 14, wherein the gel composition comprises an annular body that separates the interior space into first and second chambers.

17. A method of separating a fluid sample into component fractions having different densities, comprising:
providing a vessel that defines an interior space and includes a gel composition disposed in the interior space, the gel composition comprising a silicone fluid;
at least two kinds of particles dispersed in the silicone fluid, at least of one said at least two kinds of particles being a nanoparticle, said at least two kinds of particles differing from one another in their surface chemistry properties;
disposing said fluid sample in the interior space adjacent the gel composition; and
moving the vessel at a velocity sufficient to cause at least one component fraction of said fluid sample to move through the gel composition to effect separation from at least one other component fraction of said fluid sample.

18. The method of claim 17, wherein one of said at least two kinds of particles is more hydrophobic than another of said at least two kinds of particle.

19. The method of claim 17, wherein moving the vessel comprising rotating the vessel.
